(19) United States
(12) Patent Application Publication
(10) Pub. No.: US 2004/0018580 A1
(43) Pub. Date: Jan. 29, 2004

(54) Synthetic substrates for prostasin

(76) Inventors: Karl X. Chai, Winter Springs, FL (US); Li-Mei Chen, Winter Springs, FL (US)

Correspondence Address: LAW OFFICES OF BRIAN S STEINBERGER, 101 BREVARD AVENUE, COCOA, FL, 32922, US

(21) Appl. No.: 10/434,323

(22) Filed: May 8, 2003

Related U.S. Application Data
(60) Provisional application No. 60/379,516, filed on May 10, 2002.

(51) Int. Cl. C12Q 1/37; C07K 7/64

(52) U.S. Cl. 435/23; 530/317

(57) ABSTRACT

Synthetic peptide substrates of prostasin, a method for synthesizing them and a prostasin assay are provided.

### FIELD OF THE INVENTION

The present invention relates to preparation of a synthetic substrate of prostasin, and claims priority from U.S. Provisional Application No. 60/379,516 filed May 10, 2002 and was funded in part by the Department of Defense Prostate Cancer Research Program Grants Number DAMD 17-98-1-8590, and DAMD 17-02-1-0032.

### BACKGROUND OF THE INVENTION

Prostasin is a serine protease known to be expressed at highest levels in the semen and prostate, however, it is also expressed in other tissues at low levels. In seminal fluid prostasin can be found in complex with a prostasin binding protein that can inhibit prostasin enzymatic activity. Prostasin mRNA is found in normal prostrate epithelial cells but is not expressed in invasive prostate cancer lines. Expression of prostasin in invasive cancer lines reduces the invasiveness of cells in vitro. Prostasin is a serine protease that may have roles in normal prostate function and in suppression of tumor cell invasion.

Prostasin expression has also been implicated in ovarian and breast cancers. It is down-regulated in invasive breast cancer cell lines.

Prostasin is also known to be an activator of the epithelial sodium channel in vitro, and is present in tissues that absorb Na+ such as the kidney, colon, lung, and salivary glands. The proper regulation of the epithelial sodium channel is crucial to maintaining sodium balance, extracellular fluid volume and blood pressure. As such, it is a protein whose regulation and expression are implicated in diseases of the kidney, hypertension, and respiratory diseases.

Prostasin may also have a role in regulating various aspects of the male reproductive system, which affect male fertility.

Prostasin's physiological functions are not well characterized, and at present, neither are its substrates, inhibitors, co-factors or other regulators. Identifying these factors would be very advantageous in the ultimate development of drug therapies, assays and diagnostic tools for diseases that relate to the regulation and function of prostasin.

Prostasin belongs to the group of proteases called “serine proteases.” Serine proteases are a family of enzymes that cut peptide bonds in other proteins. This activity is dependent on a set of amino acid residues in the active site of the enzyme, one of which is always a serine.

Serine proteases are inhibited by a group of inhibitors named serpins, so called because they are serine protease inhibitors. Structurally, serpins have a central sheet and an exposed reactive center loop (RCL) at the top of the protein that contains the target cleavage sequences. Serpins work by mimicking the three-dimensional structure of a normal substrate of the protease, and the serine protease binds the serpin instead of its normal substrate. This function alone would block any further activity by the proteases. However, the proteases also cleave the serpin, forming a covalent bond linking the molecules and making a change in the three dimensional structure of the serpin which moves the attached protease to a location where it can be destroyed.

U.S. Pat. No. 6,420,157 to Darrow, et al. describes an expression vector system which identifies modulators and substrates of prostasin. The present invention provides a method for doing so without laboratory experimentation.

### SUMMARY OF THE INVENTION

One aspect of the invention relates to substrates of prostasin.

Another aspect of the invention relates to a method for identification of amino acid sequences that are the best substrates of prostasin.

A further aspect of this invention is a method for identifying gene sequences that regulate the function of prostasin.

Further objects and advantages of this invention will be apparent from the following detailed description of a presently preferred embodiment which is illustrated schematically in the accompanying drawings

### BRIEF DESCRIPTION OF THE DRAWINGS

FIG. 1 shows the amino acid sequence for the “leu-ile-ala-arg (liar)” sequence of the invention (SEQ ID No. 1).

FIG. 2 shows the amino acid sequence for a “regular” serpin sequence (SEQ ID No 2).

FIG. 3A shows representative purification profiles for each purification step.

FIG. 3B shows proteins from each purification step subjected to SDS-PAGE/Coomassie blue staining.

FIG. 3C shows a Western blot analysis using mPBP antibody.

FIG. 4A shows a Western blot analysis using mPBP antibody and a prostasin antibody and both antibodies recognize prostasin-mPBP complex at 82 kDa.

Prostasin antibody recognizes purified prostasin but not purified mPBP. The mPBP antibody recognizes purified mPBP but not purified prostasin.

FIG. 4B shows the time course of complex formation between prostasin and mPBP.

FIG. 4C shows mPBP-prostasin complex formation inhibited in presence of serine protease inhibitor aprotinin (lane 3) PMSF (lane 4) or heparin (lane 5) or mPBP antibody (lane 1) lane 2 is prostasin-mPBP complex as a control.

FIG. 5 shows Purified mPBP trypsin digested and separated using Tricine/SDS-PAGE, and immunodetected with mPBP antibody. Band indicated with arrow sent for sequence analysis and shown to be identical to sequences between position 26 and 37 of PN-1.

FIG. 6A shows prostasin-mPBP complex with thrombin and thrombin-mPBP formation and its pH dependence.

FIG. 6B shows Heparin at 0.25 unit/reaction abolishes complex formation between prostasin-mPBP but not between thrombin-mPBP.

FIG. 7 shows inhibition of prostasin activity when incubated with mPBP at different concentrations.

FIG. 8 shows GST-mPN-1 (lane 1) and GST-hPN-1 (lane 4) form a 100 kDa complex when incubated with prostasin (lanes 2 and 5). Complex formation is inhibited by aprotinin (lanes 3 and 6).

### DETAILED DESCRIPTION OF THE INVENTION

Before explaining the disclosed embodiment of the present invention in detail it is to be understood that the invention is not limited in its application to the details of the particular arrangement shown since the invention is capable of other embodiments. Also, the terminology used herein is for the purpose of description and not of limitation.

Schecter and Berger, in their 1967 article “On the size of the active site in proteases,” Biochem. Biophys. Res. Commun. 27:157162, have established nomenclature for describing the RCL of molecules that are cleaved by serine proteases, now widely used in the literature. This nomenclature designates the amino acids on each side of the scissile bond, the place where cleavage occurs, as follows:

Amino terminus . . . P₁₅ . . . P₄P₃P₂P₁Scissile BondP′₁P′₂P′₃P′₄ . . . Carboxyl terminus, wherein P or P′ represents a single amino acid (of which there are 20, so each P and P′ has 20 possibilities), and the subscript number below the P or P′ represents the number of the amino acids from the scissile bond. (The dots indicate an interrupted amino acid sequence, for example P₁₅ is not directly adjacent to the amino terminus)

It is a novel finding of the present invention that the best substrates of prostasin are those molecules which have leu-ile-ala-arg (the “liar”) sequence in the P₁-P₄ positions respectively, hereinafter referred to as Sequence ID 1, and at the P₅-P₁₅ position do not have a sequence characteristic of serine protease inhibitors, that is, they have a random and not a “regular” serpin sequence. A “regular” serpin sequence is one in which a consensus has been determined that P₉-P₁₂ are occupied by residues with short side-chains such as alanine, glycine or serine, and P14 is usually threonine or serine and P₁₅ is usually glycine. The regular serpin sequence is hereinafter referred to as Sequence ID 2.

In classical “lock and key” theory of enzyme-substrate action, the protease is the “lock” having openings that precisely match the “key” or substrate that comes in to fit the protease. An optimally fit substrate is the best substrate and will be cleaved at the scissile bond. The fate of the protease substrate interaction is largely dependent on the types of residues at P₅-P₁₅ which determine that it is either a substrate, or an inhibitor.

Once a substrate binds to the prostasin protease via its P₁-P₄ a cleavage will occur at the scissile bond, and residues to the right of the cleavage (i.e., P′1-P′4 and beyond will dissociate from the protease. If P₅-P₁₅ are random, then the rest of the substrate will eventually dissociate from the protease regenerating an active prostasin serine protease, ready for cleavage on the next molecule. If P₅-P₁₅ conform to the serpin class of proteins while P₁-P₄ are the same as the “liar” sequences of the invention, the scissile bond will still be cleaved and the P′₁-P′₄ beyond will still dissociate, but the P₁-P₁₅ and the rest of the protein will be “stuck” on the prostasin protease, permanently disabling it from cleaving any more proteins.

Synthetic peptide substrates are widely used to assay the proteolytic activity of enzymes in vitro. They can be used to investigate the substrate specificity of a given protease, to monitor the enzyme activity during the course of purification, and to measure the residual enzyme activity when analyzing the kinetics of a reaction between a protease and an inhibitor. Synthetic substrates consist of a short peptide sequence in which the acyl group is conjugated to a chromogenic or fluorogenic group.

Chromogenic substrates generate a chromogenic compound upon cleavage. The rate of hydrolysis is measured using a spectrophotometer, either continuously or in periodically stopped assays. Commonly used chromogenic substrates are peptide 4-nitroanilides (pNA) and peptide thioesters.

Fluorogenic substrates generate a fluorogenic compound upon cleavage. The rate of hydrolysis is measured using a spectrofluorometer, either continuosly or in stopped assays. Commonly used fluorogenic substrates are MCA substrates (peptidyl 4-methyl-7 coumarylamides and naphtylamide substrates.

The following examples are provided for purposes of illustration and not limitation

### EXAMPLE 1

### Purification of a Prostasin-Binding Protein (mPBP)

Sample preparation—The procedure was carried out as described in Chen, L. M., Skinner, M. L, Kauffman, S. W., Chao, J. Chao, L. Thaler, C. D. and Chai, K. X., J. Biol. Chem 276, 21434-21442 (2001) (incorporated herein by reference) with modifications. Mouse seminal vesicle fluid was expressed in buffer A (20 mM sodium phosphate pH, 6.8) at a ratio of one pair of seminal vesicles per 1.0 ml of buffer. The sample was then centrifuged at 10,000×g for 30 mins at 4 degrees C. to remove insoluble material.

The following chromatography steps were performed at room temperature.

Anion-exchange chromatography—Two milliliters of the mouse seminal vesicle supernatant were applied onto an anion-exchange Econ-Cartridge (Q, 1 ml: Bio-Rad, Hercules, Calif.) equilibrated with buffer A, at a flow rate of 1 ml/min. After washing with 10 ml buffer A, the cartridge was eluted with 10 ml of 1 M NaCl/buffer A. Prostasin-binding activity was monitored by means of a prostasin-binding assay as described in Chen et al (2001),

Hydroxylapatite chromatography—Fractions containing the prostasin-binding activity from the Q cartridge were applied onto a hydroxylapatite Econ-Cartridge (http, 1 ml; Bio-Rad) equilibrated with buffer A. The cartridge was washed with buffer A (10 ml) and eluted with 0.2 M sodium phosphate buffer (pH 6.8, 10 ml) followed by 0.5 M sodium phosphate buffer (pH 6.8, 10 ml).

Cation-exchange chromatography—Factions containing the prostasin-binding activity from the http cartridge were diluted with buffer A and applied onto a cation-exchange Econ-Cartridge were diluted with buffer A and applied onto a cation-exchange Econ-Cartridge (CM, 1 ml; Bio-Rad) equilibrated with buffer A. After washing with buffer A, the cartridge was eluted with a 0-0.75 linear NaCl gradient in buffer A (20 ml). Fractions containing prostasin-binding activity were collected and concentrated through a Centricon-10 concentrator (Millipore, Bedford, Mass.) with several changes of PBS (phosphate-buffered saline, pH 7.4, Life Technologies, Gaithersburg, Md.) and then stored at −20 degrees C. before further characterization.

Scaled-up purification was performed as described above, except that the first anion-exchange chromatography was performed using the DEAD CL-6B agarose (2.5×20 cm, Amersham Pharmacia Biotech, Piscataway, N.J.). The subsequent purification steps were carried out by using 5-ml cartridges (Bio-Rad)

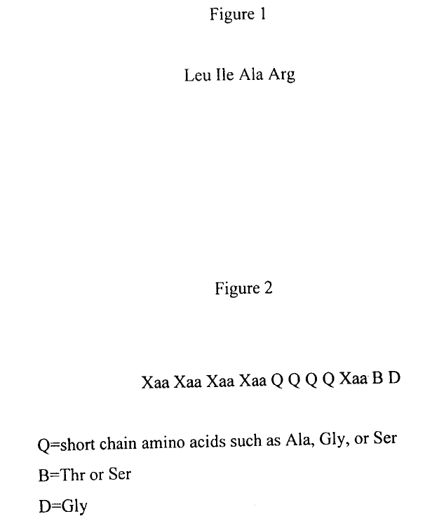

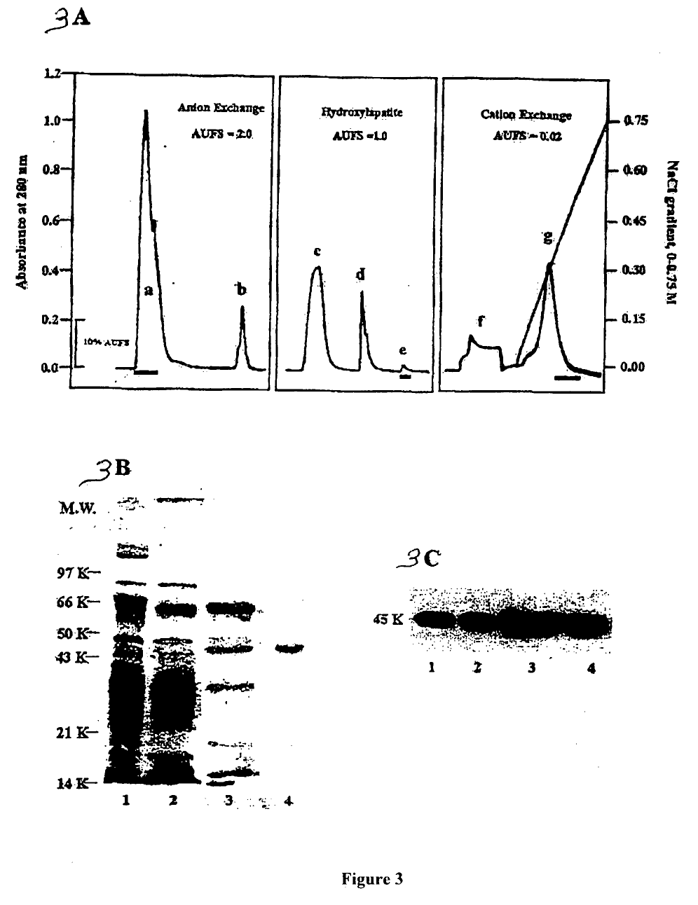

The presence of mouse prostasin-binding protein (mPBP) in each purification step was monitored using a prostasin-binding assay as described in example 2 below. FIG. 3A shows the representative purification profiles for each step. Two milliliters of mouse seminal vesicle fluid were applied onto the Q cartridge and eluted with 1 M NaCl/buffer A. Fractions were subjected to the prostasin—binding assay followed by western blotting using a prostasin antibody. The mPBP was present in flow-through fractions (peak a, indicated by the horizontal solid bar) but not the 1M naCl eluent (peak b). The mPBP-containing fractions were pooled and applied onto the http cartridge. After washing with buffer A, the cartridge was first eluted with 0.2M sodium phosphate buffer at pH 6.8 (to result in peak d) and then eluted with 0.5M sodium phosphate buffer at pH 6.8 (to result in peak e) Neither peak c (the flow-through) nor peak d contained any detectable amount of mPBP. The mPBP was detected in peak e (indicated by the horizontal solid bar). Fractions corresponding to peak e were pooled and further separated by using a CM cartridge. The CM cartridge was eluted with a linear NaCl gradient from 0 to 0.75M in buffer A. The mPBP was eluted at 0.2-0.55 NaCl buffer A (peak g, as indicated by the horizontal solid bar). The flow-through fractions (peak f) did not contain mPBP. The purified mPBP was used to generate a polyclonal antibody using rabbit as the host. (see example 2 below) Proteins from each purification step were subjected to SDS PAGE/Coomassie blue staining (FIG. 3B) or western blot analysis using the mPBP antibody (FIG. 3C) As shown in FIG. 1B, the purified mPBP (lane 4, 5 micrograms) migrated at '45 kDa in an SDS-PAGE under reducing conditions. As shown in FIG. 3C, the mPBP antibody recognized a 45 kDa protein in mouse seminal vesicle fluid, as well as the purified mPBP itself.

### EXAMPLE 2

### Identification of mPBP as Protein Nexin-1

Preparation of Polyclonal Antiserum

An anti-serum against PBP was prepared according to the procedure described in Chen et al. Briefly, 0.5 ml of the purified mPBP (250 micrograms) was emulsified with an equal volume of complete Freund's adjuvant (Sigma-Aldrich, St. Louis, Mo.) and injected subcutaneously into a 1.5 kg New Zealand White female rabbit (Charles River Laboratories, Wilmington, Mass.). Booster injections were made with 100 micrograms of mPBP (emulsified with incomplete Freund's adjuvant, Sigma-Aldrich) for 3 times at 3-week intervals. Pre-immune rabbit serum was collected before the initial immunization.

Prostasin-binding Assay and Western Blot Analysis

The procedures were performed according to Chen et al. Briefly, purified recombinant prostasin was incubated with samples from each purification step or the final purified mPBP at 37 degrees C. for 1 hour or for various times as indicated. The binding reaction was stopped by the addition of SDS sample buffer (1×SDS sample buffer=62.5 mM Tris-Hcl at Ph 6.8, 2% (v/v) glycerol, 2% SDS (w/v) and 2% Beta-mercaptoethanol). The reaction mixtures were then boiled for 5 min, and resolved in 10% SDS-polyacrylamide gels. The resolved proteins were then transferred to nitrocellulose membranes, and analyzed with either a prostasin antibody or the mPBP antibody. Signals were detected using an ECL detection procedure with the WestPico reagents (Pierce, Rockford, Ill.) following the manufacturer's protocol. The membrane was then exposed to X-ray film (Midwest Scientific, St. Louis, Mo.) The prostasin antibody was used at 1:2,000 dilution, the mPBP antibody was used at 1:10,000 dilution, and the secondary antibody (goat anti-rabbit IgG, Sigma-Aldrich) was used at 1:10,000 dilution. All antibodies were diluted in 5% non-fat milk in TBS-T (TBS-T=20 mM Tris-Hcl at pH 7.6, 0.14M NaCl and 0.1% Tween-20).

Amino Acid Sequence Analysis

The purified mPBP (6 micrograms) was incubated with various amounts of trypsin (LifeTechnologies) at 37 degrees C. for 30 minutes, and subjected to Tricine/SDS-PAGE (Schagger, H., and von Jagow, G. (1987) Anal. Biochem. 166, 368-379.) followed by transferring to the Immobilon-P membrane (Fisher, Pittsburgh, Pa.). One membrane was subjected to immunodetection with the mPBP antibody, an identical membrane was stained with 0.02% Coomassie blue R-250 in 40% methanol and 5% acetic acid for 30 seconds. The membrane was then destained in 40% methanol and 5% acetic acid for 1 min, rinsed in distilled water for 3×5 min to remove the destaining solution, and air dried. A stained band at 10 kDa, which was recognized by the mPBP antibody, was for amino acid sequence analysis at the Protein Core Facility of the University of Florida (Gainesville, Fl)

Enzymatic Assay

Recombinant human prostasin was purified as described in Chen et al. A synthetic substrate, N-t-Boc-Gln-Ala-Arg-7-amido-4-methyl coumarin (QAR-AMC) was purchased from Sigma-Aldrich. The purified mPBP (concentration range 0-0.4 micromolar) was incubated with prostasin (0.8 micromolar) for 30 mins at 37 degrees C. The binding reaction mixture (20 microliters) was then added to 80 microliters of 50 micromolar Tris-HCL) pH 8.0/0.1% bovine serum albumin containing the QAR-AMC substrate (final concentration: 100 micromolar) in 96-well microtiter plates (Costar 3903, Cambridge, Mass.). The velocity of substrate hydrolysis was measured using a Wallac 1420 Victor mulitlabel counter at wavelength 355 nm and wavelength 460 nm. The residual activity of prostasin (velocity of the inhibited enzyme reaction/velocity of the uninhibited enzyme reaction) as plotted versus the mPBP concentration.

Molecular Cloning, Expression, and Purification of Recombinant Mouse and Human Protease Nexin-1 (PN-1, Spi-4)

A cDNA encoding the mature peptide of mouse protease nexin-1 (PN-1 or Spi-4) was cloned from mouse seminal vesicle mRNA by reverse-transcription-polymerase chain reaction (RT-PCR). Total RNA of mouse seminal vesicle was isolated using a procedure described in Chirgwin, J. M., Przybyla, A. E., MacDonald, R. J. and Rutter, W. J. (1979) Biochemistry 18:5294-5299 (incorporated herein by reference.), an Oligotex mRNA Mini Kit from QIAGEN (Valencia, Calif.) was used to isolate the mRNA. The following oliognucleotide primerswere used in the RT-PCR to generate the mouse PN-1 cDNA: upstream: 5′GGAATTC TCC CAG TTC AAC TCT CTG TC-3′ and downstream: 5′-CCGCCTCGAG TCA GGG CTT GTT CAC CTG GC-3′. The underlined sequences are adapters for restriction enzyme sites. The mouse PN-1 specific sequences were derived from the Genbank™ mouse PN-1 sequence of X70296. The upstream primer sequence corresponds to base numbers 206-225 of X70296, the first codon (206-208) is that of Ser, the amino-terminal residue of the mature mouse PN-1 peptide (Vassalli, 1993) The downstream primer sequence corresponds to base numbers 1,323-1,342 including the terminationcodon (1,340-1,342). The RT-PCR was performed as described in Chen, L. M., Hodge, G. B., Guarda, L. A., Welch, J. L., Greenberg, N. M. and Chai, K. X. (2001), and incorporated herein by reference using 3 micrograms of mouse seminal vesicle mRNA as the template. A single cDNA band was amplified. The Taq DNA polymerase was removed by phenol/chloroform extraction, and the cDNA was treated with EcoR I and Xho I under proper buffer conditions. The restriction-modified cDNA was then inserted into the p'GEX-6P-1 vextor (Amersham Pharmacia Biotech) at the corresponding sites, resulting in a fusion gene construct that encodes GST-mPN-1 (GST: glutathione-S-transferase) The amplified PN-1 portion of the fusion gene was completely sequenced, no error in the PN-1 sequence was found. A human PN-1 c-DNA encoding the mature peptide was cloned from the total RNA of the human breast carcinoma cell line MDA-MB-435s (American Type Culture Collection, Manassa, Va. essentially as described above, resulting in the GST-hPN-1 construct. The primers used for the cloning wee: upstream: 5′GGAATTC TCC CAC TTC AAT CCT CTG TC-3′, downstream: 5′GGAATTC TAC GGG TTT GTT TAT CTG CC-3′/ The underlined sequences are adapters for restriction enzyme sites. The human PN-1 specific sequences were derived from the GenBank™ human PN-1 sequence of A03911. The upstream primer sequence corresponds to base numbers 82-101 of A03911, the first codon (82-84) is that of Ser, the amino terminal residue of the mature human PN-1 peptide. (Scott, R W., Bergman, B. L., Bajpai, A, A. Hersh, R. T, Rodriguez., H., Jones, B. N. Barreda, C., Watts, S. and Baker, J. B. (1985) J. Biol Chem 260:7029-7034). The downstream primer sequence corresponds to base numbers 1,199-1,218, including the termination codon (1,216-1,218). The GST-mPN-1 and the GST-hPN-1 constructs were then transformed into the TOPP-10 strain of E. coli cells (Stratagene, LaJolla, Calif.) For production of the recombinant fusion proteins, cells harboring the constructs were frown to an optical density of 0.8 (at 600-nm wavelength) and recombinant protein expression was induced with 0.2 mM of IPTG (isopropylthio-beta-galactoside) at 37 degrees with shaking at 250 rpm for 1 h. Cells were collected by centrifugation at 4000 rpm for 20 minutes at 4 degrees C. washed in 1×PBS (pH 7.4) and re-centrifuged with the same settings. GST-mPN-1 or GST-gPN-1 was then purified by glutathione-agarose affinity chromatography using protocols recommended by the manufacturer (Amersham Pharmacia Biotech).

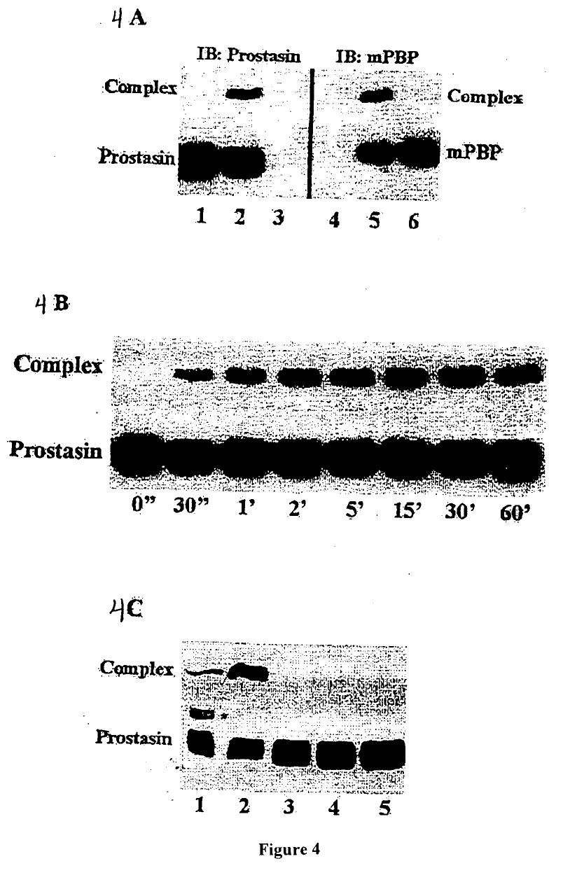

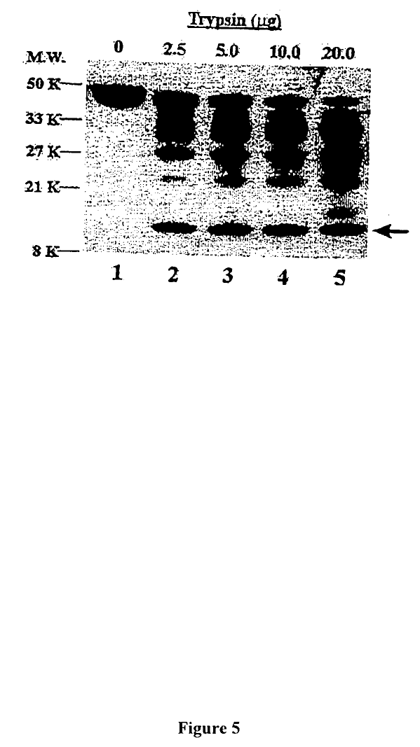

The purified mPBP was tested for its biochemical activities using the prostasin binding assay. Complex formation between mPBP and purified prostasin was analyzed by western blotting using the mPBP antibody and a prostasin antibody. Both antibodies recognized the complex of prostasin-mPBP at 82 kDa (FIG. 4A, lanes 2 and 5) The prostasin antibody also recognized the purified prostasin (FIG. 4A, lane 1) unbound prostasin (FIG. 4A, lane 2) but not the purified prostasin (FIG. 2, lane 6) unbound mPBP, but not the purified prostasin (FIG. 4, lane 4) FIG. 4B shows the time-course of complex formation between prostasin and mPBP. The complex was detected after 30 seconds of incubation and progressed during the incubation time course (60 minutes). As shown in FIG. 4C, the complex formation was inhibited in the presence of the serine protease inhibitor aprotinin (lane 3) or PMSF (lane 4) or heparin (lane 5) or the mPBP antibody (lane 1). The complex formation of prostasin and mPBP was used as a control (FIG. 4C, lane 2).

To reveal the identity of mPBP the purified mPBP was subjected to a trypsin digestion and the digested mixture was separated using Tricine/SDS-PAGE. After transferring the resolved samples to the Immobilon-P membrane, one set of the samples was immunodetected with the mPBP antibody. In the sample without trypsin digestion (FIG. 5, lane 1) the mPBP antibody recognized a single band at 45 kDa. With increasing amounts of trypsin added in the digestion mixture, several mPBP immunoreactive bands wee detected (FIG. 5, lanes 2-5) In particular, a 10 kDa band (indicted by an arrow) was separated furthest in the gel from other bands. The corresponding band in a set of the exact samples transferred to an Immobilon-P membrane, stained with Coomassie blue, was sent for amino acid sequence analysis. The amino-terminal sequence of this fragment was determined to be SLEELGSNTGIQ, which is identical to the sequences between position 26 and 37 of the GenBank mouse protease nexin-1 (PN-1) translated sequence (accession number X70196). This result suggests that PBP may be identical to PN-1, a serine protease inhibitor (serpin).

Prostasin Binding Protein Inhibits Prostasin's Activity

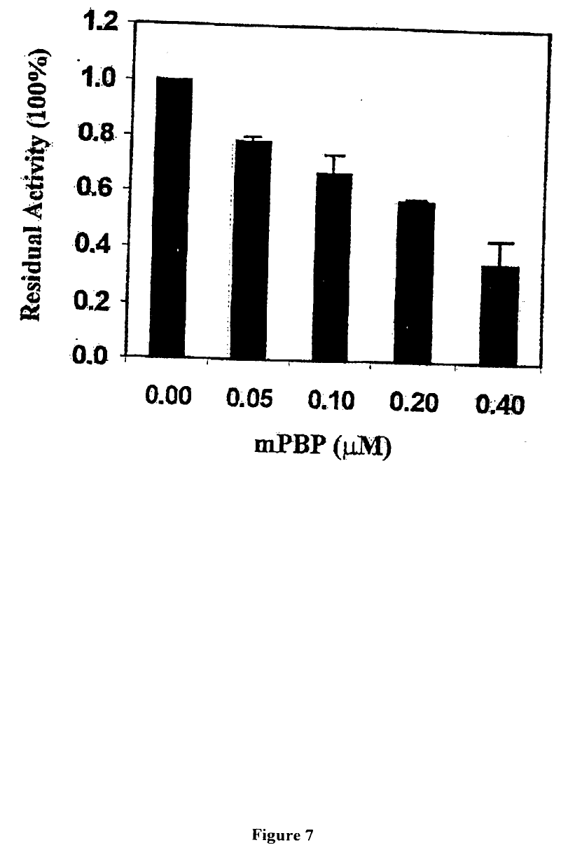

The prostasin-binding protein that was identified in the seminal vesicle inhibits prostasin's activity as determined by membrane-overlay zymography. QAR-AMC was used as a substrate for prostasin to test the inhibitory activity of the purified mPBP. Pprostasin (0.8 micromolar) was incubated with 0, 0.05, 0.1, 0.2 or 0.4 micromolar mPBP at 37 degress C. for 30 mins. The reaction mixture was then to the assay buffer containing a final concentration of 100 micromolar QAR-AMC substrate. As shown in FIG. 7, when incubated with mPBP at different concentrations, prostasin's activity was inhibited in a dose dependent manner.

Prostasin Forms a Complex with Recombinant Protease Nexin-1

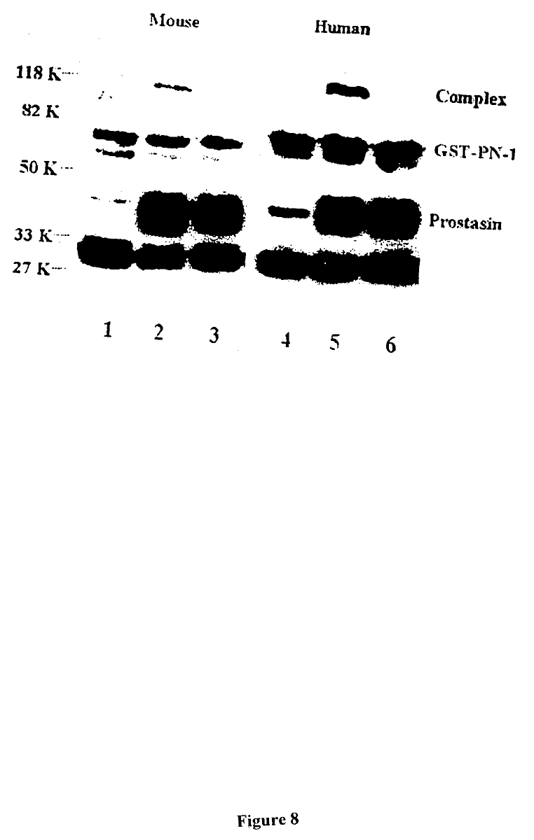

To establish if mPBP is indeed protease nexin-1, mouse and human protease nexin-1 (PN-1) cDNA were cloned into the pGEX-6P-1 expression vector. The recombinant protein products have the schistosomal glutathione-S-transferase (GST) fused to t he N-terminus of the PN-1. The GST fusion proteins were affinity-purified using glutathione-conjugated agarose-beads. For each type of recombinant protein, cleared supernatant of cell lysate from one liter of culture was incubated with 1 ml of 505 glutathione-beads. The beads were eluted with 1 ml of fresh 10 mM glutathione in 50 mM Tris-Hcl, pH 8.0. Twenty microliters of the eluent were incubated with 0.5 micrograms of recombinant prostasin at 37 degrees C. for 60 minutes in the absence or presence of aprotinin. Both the GST-mPN-1 (FIG. 8, lane 1 alone at 64 kDa) and the GST-hPN-1 (FIG. 8, lanes 2 and 5). The complex formation was inhibited by the serine protease inhibitor aprontinin (FIG. 8, lanes 3 and 6) These results further indicate that the mPBP is the serpin protease nexin-1. for this immunoblot, both the prostasin antibody and the mPBP antibody were used as the primary antibody, and a goat anti-rabbit IgG conjugated with HRP was used as a secondary antibody. An immunoreactive band at 30 kDa and two other minor immunoreactive bands at 42 kDa and 60 kDa were likely the products of non-specific degradation of the recombinant PN-1, since no protease inhibitors were added in the cell lysate during purification.

### EXAMPLE 3

### Identification of the Reactive Sequence of the Serpin PN-1

The reactive site sequences of PN-1 at locations P₁-P₄ are known to be Leu-Ile-Ala-Arg. It has been shown with the serpin Kallistatin that its reactive site sequence was also the best for a substrate.

### EXAMPLE 4

### Heparin and Thrombin Binding to Pn-1

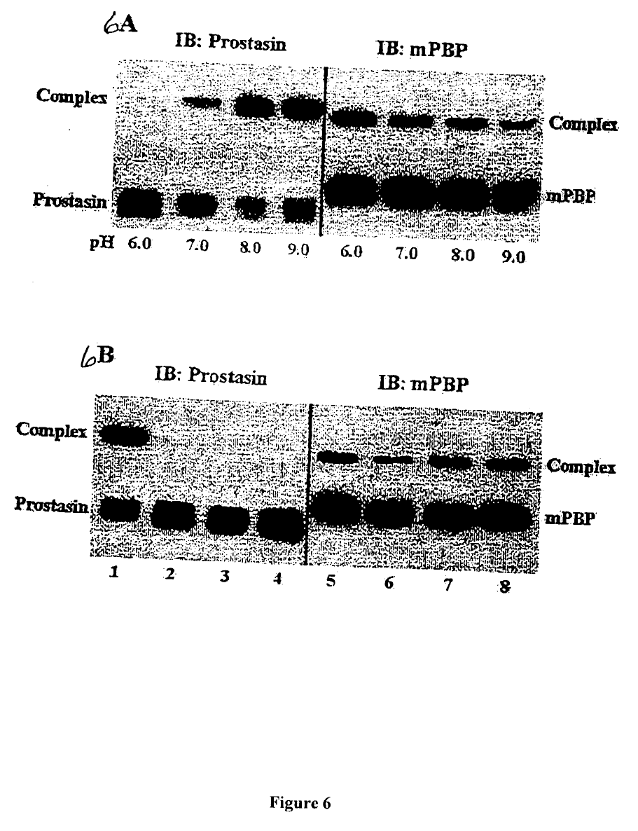

It has been known that PN-1 can form complexes with various serine proteases, including thrombin. FIG. 6A shows the complex formation between prostasin-mPBP and that between thrombin-mPBP. We also tested if the complex is pH dependent because prostasins's optimum pH is 9.0. The results showed that the purified mPBP formed a complex with prostasin as well as thrombin. The complex formation of mPBP and prostasin is increased with higher pH in the binding conditions while the complex formation of mPBP and thrombin is somewhat decreased with increasing pH. We further tested if heparin may have a different effect on the complex formation between prostasin-mPBP versus on that between thrombin-mPBP. In FIG. 6B, its is shown that heparin, at 0.25 unit/reaction, completely abolished the complex formation between prostasin—mPBP but not between thrombin-PMP.

I A heparin binding site has been mapped in PN-1 by Stone, S. R. Brown-Luedi, M. L., Rovelli, G. Guidolin, A. McGlynn, E. and Monard, D. (1994) Biochemistry 33:7731-7735. In the presence of heparin, the inhibitory activity of PN-1 to several serine proteases, such as thrombin and factor Xa is known to be enhanced. In the presence of heparin, however the binding between prostasin and PN-1 is abolished Also, in an enzymatic assay using the QAR-AMC substrate, pre-incubation of mPBP and heparin was able to prevent prostasin inhibition by mPBP. This is a novel finding of PN-1's serine protease inhibition mechanism, having potentially profound implications, especially in cancer biology. PN-1 can bind to heparin-like molecules, or heparan sulfate proteoglycans (HSPG) on the cell surface and this binding apparently accelerates thrombin inhibition by PN-1. The HSPG, as a component of the extracellular matrix (ECM) is suggested to play a major role in cell-matrix signaling. Since prostasin is a GPI-anchored membrane protease which has an anti-invasive activity in vitro, it is likely that prostasin's anti-invasion activity is regulated by PN-1 and the ECM in the tissue microenvironment. Both the membrane bound and secreted prostasin may be a proteolytic regulator of cell surface events but may also serve as a receptor or a ligand in ECM signaling or tissue remodeling under physiological or pathological conditions. The complex formation between PN-1 with two of its target enzymes, prostasin and thrombin, is affected by pH (FIG. 6) Changes in intracellular pH have been shown to be a mechanism of cell signaling.

While the invention has been described, disclosed, illustrated and shown in various terms of certain embodiments or modifications which it has presumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

We claim:

1. An synthetic substrate of prostasin, prepared by obtaining a peptide sequence of a natural substrate wherein the reactive center loop of the substrate comprises an amino acid sequence wherein
P₁₅ P₁₄P₁₃P₁₂P₁₁P₁₀P₉P₈P₇P₆P₅P₄P₃P₂P₁, are amino acid residues, P₁-P₄ comprises SEQ ID 1 and P₅-P₁₅ is not SEQ ID 2, and conjugating said protein sequence to a chromogenic or fluorogenic group.

2. A synthetic substrate, as in claim 1, wherein said P₅-P₁₅ amino acids residues are removed.

3. A synthetic substrate, as in claim 1, wherein said peptide sequence is conjugated to a chromogenic group.

4. A synthetic substrate, as in claim 2, wherein said peptide sequence is conjugated to a fluorogenic group.

5. A synthetic substrate, as in 2 claim, wherein said peptide sequence is conjugated to a fluorogenic group.

6. A prostasin enzyme assay comprising a synthetic prostasin substrate created by obtaining a protein containing the sequence of a natural substrate wherein the reactive center loop of the substrate comprises
P₁₅ P₁₄P₁₃P₁₂P₁₁ P₁₀P₉P₈P₇P₆P₅P₄P₃P₂P₁ wherein P is an amino acid residue, P₁-P₄ comprises SEQ ID 1 and P₅-P₁₅ is not SEQ ID 2 and conjugating said peptide sequence to a chromogenic or fluorogenic group to provide a synthetic substrate, reacting said synthetic substrate with prostasin, and measuring the resulting concentration of chromogenic material with a spectrophotometer or a spectrofluorometer.

7. An assay, as in claim 6, wherein said amino acid residues P₅-P₁₅ are removed.

8. An assay, as in claim 6. wherein said concentration of chromogenic material is measured with a spectrofluorometer.

9. An assay, as in claim 6, wherein said concentration of chromogenic material is measured with a spectrophotometer.

10. An assay, as in claim 6 wherein inhibitors of prostasin are included in the assay, and the degree to which the inhibitor decreases the function of prostasin is measured by assessing the decrease in concentration of chromogenic compounds.

11. An assay, as in claim 6, wherein the inhibitors of prostasin comprise compounds wherein
P₁₅ P₁₄P₁₃P₁₂P₁₁P₁₀P₉P₈P₇P₆P₅P₄P₃P₂P₁, are amino acid residues, P₁-P₄ comprises SEQ ID 1 and P₅-P₁₅ comprises SEQ ID 2.

12. An assay, as in claim 6, wherein said synthetic substrate is bound to a solid surface.
